(19) United States
(12) Patent Application Publication
(10) Pub. No.: US 2024/0218434 A1
(43) Pub. Date: Jul. 4, 2024

(54) PCR SYSTEM

(71) Applicant: Hewlett-Packard Development Company, L.P., Spring, TX (US)

(72) Inventors: Michael Cumbie, Corvallis, OR (US); Brian J Keefe, San Diego, CA (US); Kenza Samlali, Montreal (QC); Viktor Shkolnikov, Palo Alto, CA (US)

(73) Assignee: Hewlett-Packard Development Company, L.P., Spring, TX (US)

(21) Appl. No.: 18/557,281

(22) PCT Filed: Apr. 30, 2021
(86) PCT No.: PCT/US2021/030085

§ 371 Date: Oct. 25, 2023

(51) Int. Cl. C12Q 1/6844; B01L 3/00; B01L 7/00

(52) U.S. Cl. CPC C12Q 1/6844; B01L 3/502715; B01L 3/502792; B01L 7/52; B01L 2200/16; B01L 2300/0645; B01L 2300/0654; B01L 2400/0427

(57) ABSTRACT

A PCR system is described. The PCR system comprises a thermocycling chamber comprising an electrode assembly configured to generate an electric field and cause reversible electrowetting of a PCR mixture within the thermocycling chamber; and an optical sensor configured to obtain optical signals from the thermocycling chamber. Also described is a method of manufacturing a PCR system and a method of performing PCR.

### BACKGROUND

The Polymerase Chain Reaction (PCR) is used to amplify specific nucleic acid sequences and detect their presence in a sample. PCR can be used for many different applications, including quantification of gene expression, patient genotyping and also as a diagnostic tool to identify the presence of one or more pathogens, for example bacteria or viruses in a sample from a patient by amplifying and detecting nucleic acid sequences that are specific to a particular pathogen. Personalised medicine requires genotyping using PCR in which the detection of one or more biomarkers, for example specific mutations, may influence clinical decisions on the nature or type of medical intervention.

PCR subjects a sample to amplification conditions in the presence of an enzyme capable of elongating nucleic acid strands, for example a polymerase. The three basic steps of a single round or cycle of PCR amplification are denaturation, annealing and chain extension, each optimally taking place at different temperatures (typically 94-98° C. for denaturation; 50-65° C. for annealing, and 70-80° C. for chain extension, depending on polymerase), with each set of three steps being known by the term “thermocycling”. The amplification products (amplicons) are detected optically, typically using fluorescent reporters.

### BRIEF DESCRIPTION OF THE DRAWINGS

FIG. 1A is a plan view of a thermocycling chamber of an example PCR system;

FIG. 1B is a side view of the thermocycling chamber of FIG. 1A;

FIGS. 1C and 1D show plan and side views of the thermocycling chamber of FIGS. 1A and 1B containing a sample;

FIGS. 1E and 1F show plan and side views of the thermocycling chamber of FIGS. 1A and 1B containing a sample;

FIGS. 2A and 2B show a side and plan view of a thermocycling chamber of an example PCR system; and FIGS. 2C and 2D show a side and plan view of the example thermocycling chamber of FIG. 2A being filled with a sample; and

FIG. 3 shows an example microfluidic device for use in an example PCR system.

### DETAILED DESCRIPTION

Before particular embodiments of the present method and other aspects are disclosed and described, it is to be understood that the present method and other aspects are not limited to the particular process and materials disclosed herein as such may vary to some degree. It is also to be understood that the terminology used herein is used for the purpose of describing particular embodiments only and is not intended to be limiting, as the scope of the present method and other aspects will be defined only by the appended claims and equivalents thereof.

In the present specification, and in the appended claims, the following terminology will be used:

The singular forms “a”, “an” and “the” include plural referents unless the context clearly dictates otherwise. Thus, for example, reference to “a sensor” includes reference to one or more of such sensors.

The terms “about” and “approximately” when referring to a numerical value or range is intended to encompass the values resulting from experimental error that can occur when taking and/or making measurements.

Concentrations, amounts, and other numerical data may be presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of approximately 1 wt. % to approximately 20 wt. % should be interpreted to include not only the explicitly recited concentration limits of 1 wt. % to approximately 20 wt. %, but also to include individual concentrations such as 2 wt. %, 3 wt. %, 4 wt. %, and sub-ranges such as 5 wt. % to 10 wt. %, 10 wt. % to 20 wt. %, etc.

In the following description, for purposes of explanation, numerous specific details are set forth in order to provide a thorough understanding of the present apparatus and methods. It will be apparent, however, to one skilled in the art, that the present apparatus and methods maybe practiced without these specific details. Reference in the specification to “one example” or “an example” means that a particular feature, structure, or characteristic described in connection with the example is included in at least one example. The appearance of the phrase “in one example” in various places in the specification are not necessarily all referring to the same example.

Unless otherwise stated, any feature described herein can be combined with any aspect or any other feature described herein.

As used herein, the abbreviations “PCR”, “dNTPs” and “primers” refer to the “Polymerase Chain Reaction” and its components. Specifically, the term “dNTP” refers to the 2′-deoxynucleotide triphosphates used in PCR. The four standard dNTPs are 2′-deoxyadenosine 5′-triphosphate, 2′-deoxyguanosine 5′-triphosphate, 2′-deoxycytosine 5′-triphosphate and thymidine 5′-triphosphate (already lacking a 2′-hydroxyl), though modified dNTPs incorporating labels or reporter molecules, or reactive moieties may also be used.

As used herein, the term “primer” refers to a short single stranded nucleic acid, typically an oligodeoxynucleotide (also referred to as an oligonucleotide herein), of about 15 to 30 nucleotides in length. A primer is designed to base pair in a specific or complementary manner to a nucleic acid sequence of interest, and so is considered specific to that nucleic acid. DNA is directional, with the 3′ end of one strand forming base pairs with the 5′-end of the counter strand and a primer is usually designed so that its 5′-end base pairs to the 3′-end of the nucleic acid of interest so that DNA synthesis (which occurs in a 5′ to 3′ direction) to elongate the primer can occur.

As used herein, the terms “oligonucleotide pair”, “oligonucleotide primer pair” and “primer pair” refer to a set of two oligonucleotides that can serve as forward and reverse primers for a nucleic acid of interest. As both strands are copied and amplified in a PCR reaction, each strand requires a primer: the forward primer attaches to the start codon of the template DNA strand (the anti-sense strand), while the reverse primer attaches to the stop codon of the complementary strand of DNA (the sense strand). The 5′-end of each primer binds to the 3′-end of the complementary DNA strand of the nucleic acid of interest.

As used herein, the term “nucleic acid of interest”, or “target”, refers to a polynucleotide sequence, typically of at least one hundred, two hundred, three hundred, four hundred, five hundred or up to one thousand nucleotides in length. The polynucleotide sequence may be specific to a particular organism such as a pathogen, or may be suspected of having a particular mutation along its length, and will encode a particular polypeptide or protein, or mutant form thereof. For example, the polynucleotide sequence may encode the spike protein of SARS-CoV-2, or may encode a mutant form of the epidermal growth factor receptor (EGFR) the presence or absence of which renders a patient more or less likely to respond well to cancer treatments such as erlotinib or gefitinib.

The three basic steps of a single round or cycle of PCR amplification are denaturation, annealing and chain extension, each optimally taking place at different temperatures (typically 94-98° C. for denaturation; 50-65° C. for annealing, and 70-80° ° C. for chain extension, depending on polymerase), hence the term thermocycling. The denaturation step separates the two strands of double-stranded DNA, with each strand acting as a template in the later chain extension step in which a complete complementary strand to the template is produced. An oligonucleotide primer (typically comprising 15 to 30 nucleotides to ensure a balance of good specificity and efficient hybridization) is annealed to the 3′-end of each single stranded DNA molecule, and acts as a template for the synthesis of the new strand. A DNA polymerase, and a mix of dNTPs then synthesize the new strand in the chain extension step, using the original single strand of DNA as its template. Since both strands of the original DNA duplex are used as templates, a singe round or cycle of PCR results in a doubling of the number of DNA duplexes. The number of copies thus increases exponentially with the number of cycles of amplification: after 2 cycles, four DNA duplexes are present in the sample, while after 3 cycles, 8 duplexes are present.

PCR is typically done on a prepared sample of 10-50 UL and quantified by monitoring the fluorescence of the fluid as it is thermally cycled. Since the fluorescence is proportional to the amount of nucleic acid (double stranded DNA), the fluorescence intensity increases as the number of cycles of amplification (the amount of double stranded DNA produced) increases. However, in order to achieve a high enough signal to noise ratio, typically 40 cycles are required. PCR reaction mixtures are aqueous solutions and so have a high specific heat capacity, meaning that increasing (or ramping up) the temperature of a reaction droplet in the transition from annealing (50-65° C.) to chain extension (70-80° C.), requires energy input and/or time and cooling down the solution requires time, leading to long overall test times (30 minutes or more). Higher thermal ramp rates (rapid increases or decreases in temperature) can be achieved using thin films of sample as the time taken to oscillate a liquid sample between two temperatures is proportional to the square root of the liquid depth. For example, a single thermal cycle for a thin film or shallow film of less than 200 μm is approximately 10-fold faster than the equivalent volume of liquid in a substantially spherical droplet.

As used herein, “electrowetting a liquid” refers to an electrowetting process in which a liquid volume has formed a thin film by substantially or entirely wetting the surface on which it is located, for example a surface of a thermocycling chamber, under the control of an electrical field. For example, an electrowetted thin film of liquid may refer to a liquid sample having a contact angle with the underlying surface, for example a dielectric layer or a hydrophobic layer as described herein, of less than about 45°, for example less than about 30°, for example less than about 20°, for example less than about 10°, for example less than about 5°. As used herein, the term “beaded droplet” may refer to a liquid volume having a contact angle with the underlying surface, for example a dielectric layer or a hydrophobic layer as described herein, of greater than 45°, for example greater than 50°, for example greater than 60°, for example greater than 70°, for example greater than 80°, for example greater than 90°, for example greater than 100°. The contact angle of a liquid on a surface can be measured optically, for example using a contact angle goniometer. As used herein, the term “reversible electrowetting” refers to fact that the electrowetting process can be switched on or off under the control of the electrical field, i.e. is reversible. That is, the electrowetting phenomenon is switched on by application of an electrical field, but can be reversed by at least partially switching off the electrical power that generated the electrical field. As explained above, a liquid “electrowets” a surface to form an electrowetted thin film when the electrical field is applied, and reverses its electrowetting to form a shape that more resembles a beaded droplet than a thin film in the absence of the electrical field or when the strength of the electrical field is reduced.

As used herein, the terms “capillary pressure barrier”, “capillary valve” or “capillary break”, refer to structural or material modifications used in microfluidics technologies to control fluid flow through a structure, for example a microfluidic channel, or a chamber, and function by increasing the pressure required to further advance the liquid beyond the capillary pressure barrier. This can be achieved by, for example, adjusting the depth or width of the channel or chamber, or by adjusting the contact angle of a liquid with the surface of the channel or chamber. In this way, the liquid is prevented from passing the capillary pressure barrier until an increase in injection pressure is applied to overcome the increase in capillary pressure, or until the contact angle of the liquid with the channel or chamber surface is modified (for example by temperature, as will be described below).

For PCR liquid volumes as envisaged, an electrowetted thin film may have a depth of less than 200 μm, for example less than 100 μm, for example less than 50 μm, for example less than 30 μm, for example less than 10 μm. However, thin films of liquid such as caused by electrowetting are not suited for giving high sensitivity fluorescent readings, as fluorescence intensity is proportional to the depth of the liquid sample. Rapid (<10 min), yet sensitive (<500 copies) nucleic acid detection is highly desired as it allows, for example, a patient diagnosis to be done at the point of care, accelerating medical decision making and improving patient treatment.

The present inventors have sought to develop a PCR system that enables rapid thermocycling alongside sensitive fluorescence detection, to provide sensitive and rapid readouts. The present inventors have found that it is possible to change the form or appearance of the liquid sample in a controllable manner, to enable rapid thermocycling without any loss of sensitivity during fluorescence detection. Specifically, the present inventors have found that it is possible to cause the liquid sample volume to form, by electrowetting, a thin film during thermal cycling, and to cause the liquid sample volume to form a beaded droplet during optical fluorescence detection. In other words, the depth of the liquid sample volume can be changed, depending on whether heating/cooling or fluorescence measurement is required. The present inventors have found that this control can be achieved through the use of electrode assemblies to cause the sample to reversibly electrowet the surface. By doing this, faster heating and cooling rates are achieved at the same time as not trading off optical sensitivity due to etendue of fluorescent emitter, enabling real-time quantitative RT-PCR in a quick and sensitive process.

In one example there is provided a PCR system, comprising:
a thermocycling chamber comprising an electrode assembly configured to generate an electric field and cause reversible electrowetting of a PCR mixture within the thermocycling chamber; and
an optical sensor configured to obtain optical signals from the thermocycling chamber.

In a further example there is provided a method of manufacturing a PCR system, comprising:
forming a thermocycling chamber; and
arranging an electrode assembly so as to be operable to generate an electric field within the thermocycling chamber.

In a further example there is provided a method of performing PCR, comprising:
introducing a liquid volume into a thermocycling chamber of a PCR system, wherein the liquid volume comprises a nucleic acid sample suspected of containing a nucleic acid of interest and the thermocycling chamber comprises an electrode assembly configured to generate an electric field within the thermocycling chamber;
applying a first voltage across the electrode assembly to cause the liquid volume to electrowet within the thermocycling chamber;
subjecting the liquid volume to amplification by polymerase chain reaction;
applying a second voltage across the electrode assembly to reverse the electrowetting and cause the liquid volume to form a droplet; and
detecting an optical signal from the droplet.

### PCR System

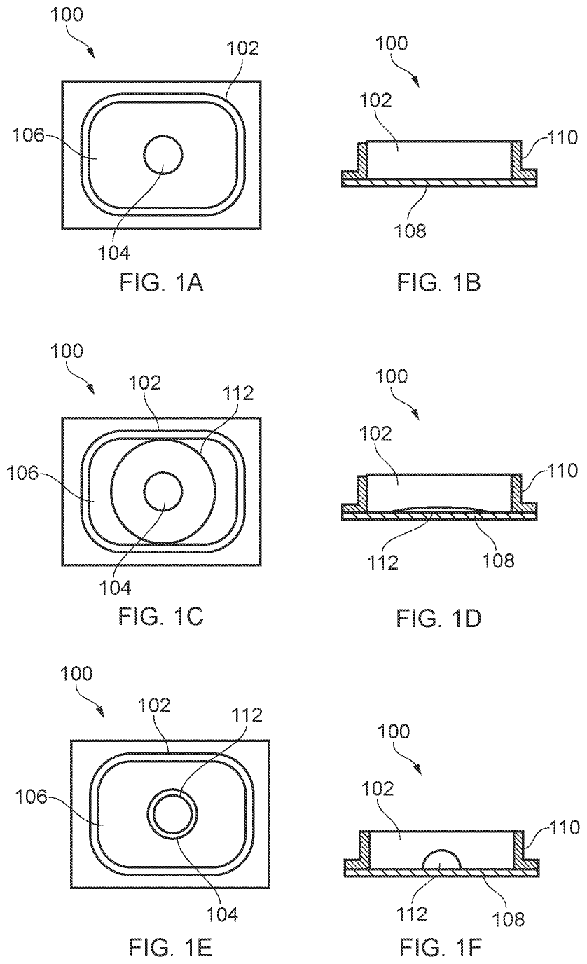

Described herein is a PCR system. The system comprises a thermocycling chamber. The thermocycling chamber comprises an electrode assembly configured to generate an electric field and cause reversible electrowetting of a PCR mixture within the thermocycling chamber; and the system further comprises an optical sensor configured to obtain optical signals from the thermocycling chamber. FIG. 1A shows in plan view a thermocycling chamber 102 of a system 100 as described herein, while FIG. 1B shows the same thermocycling chamber 102 from a side view. FIG. 1A shows an electrode assembly comprising two concentric electrodes 104 and 106, with first electrode 104 approximately circular in shape while second electrode 106 is elongate in shape. FIG. 1B shows thermocycling chamber 102 as a single well or chamber, disposed on a substrate 108 with the walls of the chamber formed by a cover layer 110 on substrate 108. For convenience, first and second electrodes 104 and 106 are not depicted in FIG. 1B.

In some examples, the PCR system comprises a single-well apparatus or a multi-well apparatus, i.e. has a single thermocycling chamber or a plurality of thermocycling chambers. In some examples, the PCR system comprises a plurality of thermocycling chambers and the plurality of thermocycling chambers are independently operable. In other words, each thermocycling chamber of the plurality of thermocycling chambers may have its own dedicated electrode assembly, so as to be independently controllable. In this way, synchronous or asynchronous control of a plurality of different PCR assays can be performed. Asynchronous control of individual thermocycling chambers of a plurality of thermocycling chambers enables the amplification of a plurality of different samples using different thermocycling, or even isothermal, protocols.

In some examples, the PCR system comprises a substrate on which the thermocycling chamber is provided or the plurality of thermocycling chambers are provided. In some examples, the PCR system comprises a substrate on which the electrode assembly and thermocycling chamber are provided. Some example substrates may include polypropylene substrates, polycarbonate substrates, polydimethylsiloxane (PDMS) substrates, silicon-based substrates, glass-based substrates, gallium arsenide based substrates, and/or other such suitable types of substrates for microfabricated devices and structures. In some examples, the substrate may be formed from a photoresist material such as SU8, an epoxy-based photoresist material, in which a pattern has been etched and into which at least a portion of the electrode assembly is to be incorporated. In some examples, the substrate is formed from a transparent material, thereby permitting optical detection from underneath the thermocycling chamber.

In some examples, the thermocycling chamber comprises an open chamber or open well. An open chamber or open well in the context of PCR technology may, for example, be a chamber with a base and side walls extending from the base, and an opening at the end of the chamber opposite to the base, such as is found on a standard 96- or 384-well plate. Open chambers or open wells allow for efficient fluid flow, as the open end of the chamber acts as a vent through which air can be expelled as a liquid volume is introduced. In some examples, the thermocycling chamber comprises a single fluid inlet, at the well opening at the top of the well with a PCR mixture being introduced via the well opening. In some examples, the thermocycling chamber comprises a single fluid inlet at the bottom of the well. In some examples, the thermocycling chamber is provided with a lid, i.e. is not an open well. In these examples, the thermocycling chamber may be provided with a separate vent or overflow chamber, accessible from the thermocycling chamber via an exhaust channel through which a liquid cannot pass, for example due to a widening of the exhaust channel acting as a capillary break or capillary pressure barrier.

In some examples, the thermocycling chamber is present on the substrate as part of a microfluidic network, in which one or more microfluidic channels are fluidically connected to the thermocycling chamber, and through which a sample for amplification and analysis are transported to the thermocycling chamber. Thus, in some examples, the substrate may be termed a microfluidic device. In some examples, the PCR system, for example a microfluidic device of the PCR system, comprises a plurality of thermocycling chambers, each fluidly connected to a central flow channel via which reagents and/or samples for amplification may be provided. In some examples, the PCR system, for example a microfluidic device of the PCR system, comprises a plurality of thermocycling chambers, each fluidly connected to its own dedicated flow channel via which reagents and/or samples for amplification may be provided. In other words, the thermocycling chamber is fluidically isolated from any other thermocycling chamber that may be present on the substrate or in the system. In some examples, the described flow channels may be provided with further electrode assemblies, operable to transport reagents and/or samples for amplification into the thermocycling chamber via electrowetting processes. In some examples, reagents and/or samples for amplification may be transported into the thermocycling chamber via injection pressure and capillary forces.

In some examples, at least a portion of the electrode assembly is disposed on the substrate. In some examples, the electrode assembly comprises two electrodes, with at least one electrode disposed on the substrate. In some examples, the electrode assembly comprises two electrodes, with both electrodes disposed on the substrate, for example in a concentric arrangement. In such examples, each electrode may be a planar or plate or pad electrode. In some examples, the electrode assembly comprises two electrodes adjacent one another on the substrate. In some examples, each electrode of a pair of adjacent electrodes is dimensioned so as to occupy half of the base of the thermocycling chamber, with the liquid volume being confined to one electrode (and one half of the thermocycling chamber) when in the droplet state, and extending over to the adjacent electrode when electrowetted. In some examples, the electrode assembly comprises two electrodes, with one electrode disposed on the substrate and the other electrode configured to contact the liquid sample from a different angle, for example from above the liquid sample. In such examples, the electrode assembly may comprise a planar electrode disposed on the substrate and a needle or pin electrode oriented into the thermocycling chamber from above. It will be appreciated that any configuration of electrode assembly that creates an electric field that passes through the interface between the liquid droplet and surrounding medium (air or oil) may be suitable.

In some examples, the electrode assembly comprises one or more planar electrodes. The one or more planar electrodes may be printed electrodes, printed onto the substrate. In some examples, the one or more electrodes may be formed from graphene, copper, silver, gold, platinum, carbon, aluminium, titanium, tantalum, aluminium-doped zinc oxide, indium-doped cadium oxide, indium zinc oxide or indium tin oxide (ITO).

In some examples, the electrode assembly comprises two concentric electrodes provided on a substrate on which the thermocycling chamber is disposed. In some examples, the concentric electrodes are embedded in the substrate such that a top surface of the substrate and the top surface of each electrode are substantially coplanar to each other. As used herein, “substantially coplanar” refers to aligning the surfaces of adjacent components with respect to each other such that transition of the liquid volume from thin (substantially wetted) film to droplet across the electrode assembly is not substantially impeded by changes in elevation. In some examples, the electrodes are separated by a gap. As will be explained further below, an electric field is applied between electrodes across the gap such that the electric field causes the liquid sample to transition from thin film to droplet across the gap based on the application of the electric field.

In some examples, the electrodes of the electrode assembly are arranged on the surface of the substrate such that the electrodes are within a predetermined distance (e.g., 10 μm) from each other. Shaping the pad topography of the first electrode enables contact angle discontinuity at the edge of the liquid volume so as to confine it to the first electrode. In some examples, a radius, length or width of the first electrode is set so as to define a desired droplet shape (for example diameter).

In another example, the electrodes may also be immediately adjacent one another. The shape of the electrodes is not limited to any particular construction disclosed herein, and can be circular, rectangular, triangular, have an irregular shape, etc.

In some examples, the electrodes of the electrode assembly are also configured to provide heat to the thermocycling chamber during the PCR amplification processes. In other examples, the thermocycling chamber is provided with a separate heat source for PCR thermocycling, for example a Peltier device disposed underneath the thermocycling chamber. A Peltier device is a solid-state active heat pump which transfers heat with consumption of electrical energy. The separate heat source may also be provided from a laser source, oven, microwave or a conductive wire.

In some examples, a PCR system comprises a thermocycling chamber provided with at least one capillary pressure barrier on an internal surface of the thermocycling chamber. The at least one capillary pressure barrier controls the location of the liquid-gas interface during filling to avoid bubble formation.

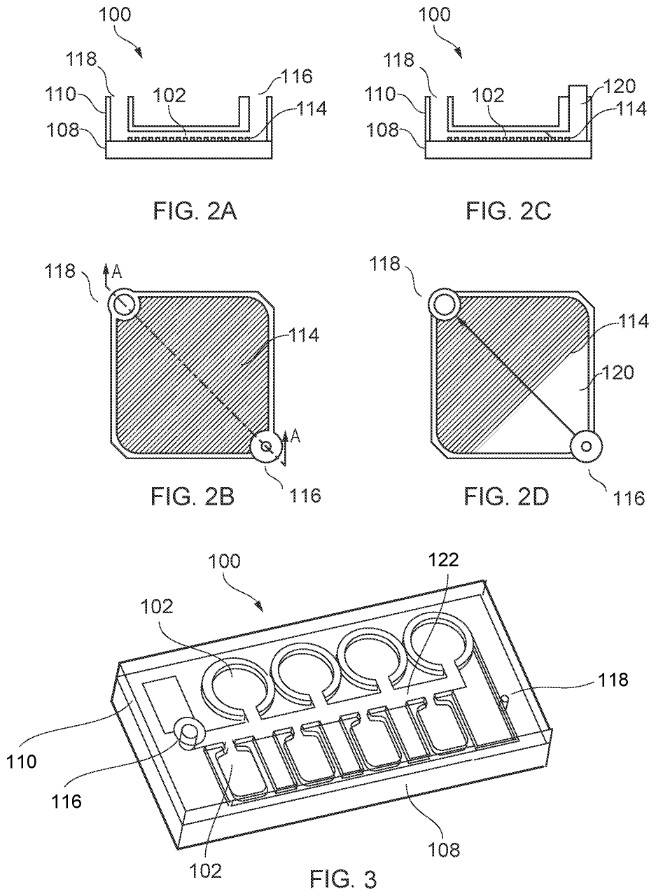

FIGS. 2A and 2B show a thermocycling chamber 102 of a PCR system 100, with like components numbered as for the preceding Figures. In FIGS. 2A and 2B, substrate 108 is provided with a plurality of capillary pressure barriers 114. For simplicity, the electrodes of the electrowetting electrode assembly are not illustrated, but it will be understood that they also would be present on or within substrate 108. Thermocycling chamber 102 is also provided with an inlet port or fluid inlet 116, and an outlet port 118, which may also be termed an exhaust or vent. In the system of FIGS. 2A to 2D, inlet port 116 and outlet port 118 are dedicated ports from above for thermocycling chamber 102, though it will be appreciated that in other configurations, a fluid inlet may for example be in the same plane as the thermocycling chamber.

In some examples, the thermocycling chamber has a high aspect ratio of at least 1:10 (height to width) and is provided with at least one capillary pressure barrier on an internal surface of the thermocycling chamber, to control the location of the liquid-gas interface during filling to avoid bubble formation. In some examples, the at least one capillary pressure barrier comprises a region of raised material on or depressed material in the floor or ceiling of the chamber. In some examples, the at least one capillary pressure barrier can be a region of the floor or ceiling of the thermocycling chamber with a different contact angle to the contact angle of the surrounding floor or ceiling, for example by depositing or printing a material having a higher hydrophobicity than the surrounding material. For example, the at least one capillary pressure barrier may be formed by depositing at pre-defined locations a hydrophobic material onto a dielectric layer. The dielectric layer and the hydrophobic material may be as described. In some examples, the at least one capillary pressure barrier comprises a resistor. In some examples, the at least one capillary pressure barrier comprises a second electrode assembly, for example a printed electrode on a PCB substrate, which is independently operable from the electrode assembly for electrowetting. In FIGS. 2A to 2D, the at least one capillary pressure barrier 114 comprises a second electrode assembly in the form of printed electrical traces on the PCB substrate, which have a raised profile above the surface of the substrate.

In some examples, the second electrode assembly functions as the at least one capillary pressure barrier to control filling of the liquid volume into the thermocycling chamber. In some examples, the second electrode assembly comprises one or more electrodes that extend across the entire width of the thermocycling chamber perpendicular to the direction of filling (that is, perpendicular to flow axis from an inlet to an outlet). In some examples, the second electrode assembly comprises a plurality of connected printed electrical traces, which function as the at least one capillary pressure barrier. In some examples, the second electrode assembly comprises a plurality of independently operable printed electrical traces, each of which functions as a capillary pressure barrier. In some examples, each printed electrical trace of the independently operable printed electrical traces extends across the entire width of the thermocycling chamber perpendicular to the direction of filling.

In some examples, the thermocycling chamber is provided with one or more capillary pressure barriers on an internal surface thereof. In some examples, the thermocycling chamber is provided with one or more thermally controllable capillary pressure barriers on an internal surface thereof. In some examples, the at least one capillary pressure barrier is thermally activated or controllable, thereby providing control over when an infilling liquid is able to pass or burst the at least one capillary pressure barrier. In some examples, the at least one capillary pressure barrier is thermally activated via optical means, for example via selective absorption of radiation from an optical source. For example, the at least one capillary pressure barrier may be formed of a material that selectively absorbs light of a particular wavelength and thereby generates heat. Selective absorber materials include ceramic, or metal oxide, materials such as copper oxide or cobalt oxide deposited on a substrate. In some examples, when the at least one capillary pressure barrier comprises a second electrode assembly (for example a plurality of printed electrical traces) the at least one capillary pressure barrier is thermally activated by providing an electrical current to the second electrode assembly. In operation, the liquid volume enters the thermocycling chamber and the liquid-gas interface is pinned at a first capillary pressure barrier (for example the first electrical trace). A short thermal pulse quickly raises the temperature locally to the capillary pressure barrier at this interface (but negligibly elsewhere), locally reducing the surface tension, and so allowing the interface to unpin. The unpinning may initially occur at a particular section of the capillary pressure barrier and then propagate along the length of the capillary pressure barrier. The preferential unpinning at a location may be controlled by a lower difference in surface tension or a lower step height of the capillary pressure barrier. Such a mode of unpinning may be used to control how the liquid fills the region between sequential capillary pressure barriers.

The liquid volume then proceeds by capillary action to further fill the chamber, until the next trace. The next pulse then unpins the interface there and the process repeats. The pulses are timed to allow the fluid to fill the region between the traces.

In some examples, the thermocycling chamber is provided with a plurality of capillary pressure barriers on the floor of the thermocycling chamber. In some examples, the thermocycling chamber is provided with a plurality of capillary pressure barriers on the ceiling of the thermocycling chamber. In some examples, the thermocycling chamber is provided with a plurality of capillary pressure barriers on the floor and the ceiling of the thermocycling chamber. In some examples, the plurality of capillary pressure barriers on the floor and the ceiling of the thermocycling chamber are aligned opposite to each other, or in an alternating pattern. In some examples, the plurality of capillary pressure barriers are independently or commonly activated by electrical heating, or by optical heating as described herein.

In some examples, the second electrode assembly performs multiple functions, including thermal control of the filling of the thermocycling chamber, and thermal control of the liquid volume during a thermocycling reaction.

In the plan views of FIGS. 2B and 2D, it can be seen that the plurality of capillary pressure barriers are oriented perpendicular to a fluid flow, causing the infilling liquid 120 to completely fill electrowetting chamber 102 on the upstream side of each capillary pressure barrier, before a thermal pulse is applied to that capillary pressure barrier to allow infilling liquid to advance. In the side view of FIG. 2C, the front of liquid 120 can be seen pinned against the third capillary pressure barrier from the right, having advanced past two capillary pressure barriers that have received been thermally actuated to let the liquid front pass.

In some examples, a dielectric layer is disposed over the electrode assembly or electrode assemblies and substrate. The dielectric layer may bridge a transfer gap between adjacent electrodes. The dielectric layer may be spun on or sputtered onto the electrode assembly and substrate. Thus, in some examples, the electrode assembly comprises a dielectric coating of polyimide, SU-8, silicon oxide, silicon nitride, aluminium oxide, aluminium nitride or any combination/stack thereof. Another suitable material is Kapton®, which may be incorporated into a coating or stack with any of the aforementioned materials.

In some examples, a hydrophobic layer can be provided and is disposed over the dielectric layer. Thus, in some examples, the electrode assembly comprises a hydrophobic coating. In some examples, the electrode assembly comprises a hydrophobic coating of PTFE, cyanoethyl pullulan (CEP), or octadecyltrichlorosilane. The hydrophobic layer may be spun on. Other suitable materials include Cyanoresin CR-S, Dyneon THV, Cytop, Rain-X, Nevosil® Si-7100, FluoroPel. The hydrophobic layer enhances surface energy control, and facilitates a transition of the liquid volume between the thin film state and the droplet state.

In some examples, a layer of material which is dielectric and hydrophobic is disposed over the electrode assembly or assemblies and substrate. In some examples the PCR system comprises a cover layer in which the thermocycling chamber is formed. The cover layer may be disposed on the substrate or, when present, the dielectric layer or hydrophobic layer. The cover layer may be formed of any suitable material for containing PCR reactions, such as found in multi-well ANSI/SLAS plates. In some examples, the cover layer may be formed of polypropylene, polycarbonate, PDMS, polyethylene terephthalate or similar suitable materials. In some examples, the cover layer is bonded to the substrate or, when present, the dielectric layer and/or hydrophobic layer, so as to form a discrete well or thermocycling chamber in which the walls of the well define the boundary of the thermocycling chamber and the extent to which a liquid volume can spread out when forming a thin film.

In the examples in which a dielectric layer and/or a hydrophobic layer are disposed over a printed electrode assembly or trace for filling control or heating (as opposed to the electrowetting electrode assembly), it will be understood that the deposition of these layers would accentuate the raised profile of the printed electrical trace thereby providing better pinning of the interface of the infilling liquid volume.

The PCR system comprises an optical sensor configured to obtain optical signals from the thermocycling chamber. In some examples, the optical sensor is a fluorescence sensor and the optical signals are fluorescence signals. As described above, fluorescent molecules are used as reporter molecules in PCR amplification, with the fluorescence intensity proportional to the amount of amplified nucleic acid material. In some examples, the optical sensor comprises a light source and a detector, wherein the light source is for example a laser diode, or an LED, configured to emit light of a wavelength suitable to cause fluorescence of a fluorescent reporter molecule during a PCR amplification process. For example, SYBR Green I, absorbs blue light with a Amax of 497 nm, and emits green light with a Amax of 520 nm. In some examples, the detector may be a charge coupled device (CCD) or pin photodiode configured to detect the emitted fluorescent light. In some examples, the optical sensor is arranged above or below the thermocycling chamber, for example above or below a plane in which the liquid sample is being thermocycled.

In some examples, the thermocycling chamber is provided on a device, for example a microfluidic device. In some examples, the substrate on which the thermocycling chamber is provided may be termed a device or chip, for example a microfluidic device or microfluidic chip. In some examples, the PCR system comprises a device on which the thermocycling chamber is provided, and the optical sensor is provided separate to the device. In some examples, the device is provided with an optical window or opening that allows transmission of light therethrough to an optical sensor located in the PCR system but outside of the device. In some examples, the optical sensor is provided on a device on which the thermocycling chamber is provided. In some examples, the optical sensor is embedded into the lid or a wall of the thermocycling chamber.

In some example, the device on which the thermocycling chamber is provided comprises a single use or disposable device. In some examples, the device may be configured to be inserted into or received by a port in the PCR system. In some examples, the PCR system may be provided with one or more fluidic connections that are configured to engage with one or more corresponding fluidic connections in the device, to enable fluid flow from the system into the device, for example to enable transfer of a sample injected into an injection port of the system to be transferred to the thermocycling chamber. In other examples, the or each thermocycling chamber may be filled with sample prior to inserting the device into the system, for example by manual pipetting a sample solution through an inlet port such as a Luer connector or membrane valve.

FIG. 3 shows an example of a microfluidic device 100 incorporating a plurality of thermocycling chambers 102. In the example device, four thermocycling chambers are approximately circular in shape (top row), and four are elongate thermocycling chambers (bottom row), all provided in a cover layer 110 on a substrate 108, though it will be appreciated that in some examples each device may have a single thermocycling chamber. For convenience, the electrode assemblies underlying each thermocycling chamber, and any capillary pressure barriers are not shown. In the device shown, each thermocycling chamber is fluidically connected to a central flow channel 112, served by an inlet port 116 in cover layer 110, though it will be appreciated that this is only one example and that other configurations are possible. In the device of FIG. 3, each thermocycling chamber 102 is also fluidically connected to a common vent 118, also in cover layer 110, though it will be appreciated that each thermocycling chamber may have its own dedicated vent.

In examples in which the thermocycling chamber is provided on a device such as a microfluidic device, the PCR system may comprise an electrical interface, configured to contact an electrical interface provided on the device. The electrical interface on the system may be coupled to any component of the device that requires electrical current to operate. Examples of such components include the electrowetting electrode assembly, heater elements, either in flat panel form or printed conductive trace form, and actuators for controlling fluid flow within the device. In some examples, the electrical interfaces may be multi-pin input/output off board connecters, for example 44-pin connectors that enable electrical coupling of the device to a computer module of the PCR system. Each pin of the electrical interface may provide an electrical contact to a specific component of the device, such as the electrowetting electrode assembly described herein. The electrical coupling of the device to the system allows control signals from the computer module to be sent to the PCR device so that electrical current can be sent to desired modules of the device.

As noted above, the PCR system may comprise a computer control module. In some examples, the computer control module comprises a processor comprising hardware architecture to retrieve executable code from a data storage device or computer-readable medium and execute instructions in the form of the executable code. The processor may include a number of processor cores, an application specific integrated circuit (ASIC), field programmable gate array (FPGA) or other hardware structure to perform the functions disclosed herein. The executable code may, when executed by the processor, cause the processor to implement the functionality of one or more hardware components of the device and/or system such as one or more electrode assemblies and/or one or more optical detectors. In the course of executing code, the processor may receive input from and provide output to a number of the hardware components, directly or indirectly. The computer control module may communicate with such components via a communication interface which may comprise electrical contact pads, electrical sockets, electrical pins or other interface structures. In one example, the communication interface may facilitate wireless communication.

In some examples, the computer control module facilitates the introduction of a sample into the thermocycling chamber, or into multiple thermocycling chambers. For example, the computer control module may control a series of valves and pumps in the system or on the device to direct flow of a test sample or solution to the thermocycling chamber.

In some examples, the computer control module may further control the processing of a sample in a thermocycling chamber, for example by subjecting the thermocycling chamber to thermocycling conditions and by controlling reversible electrowetting of the sample. For example, the computer control module may control, through the output of control signals, the operation of one or more electrode assemblies to control whether a sample in the form of a liquid volume forms a droplet shape, or substantially wets the surface on which it is located to form a film. In other examples, the computer control module may control, through the output of control signals, the operation of one or more heaters to control the temperature and duration of heating within the or each thermocycling chamber. In other examples, the computer control module may control, through the output of control signals, the operation of the optical sensor, to monitor progress of the PCR amplification and to detect the presence of a nucleic acid of interest in a sample. As a result, a sample may undergo various selected reactions, various selected heating cycles and various sensing operations under the control of the computer control module.

### Method of Manufacturing a PCR System

In some examples, a method of manufacturing a PCR system is described, comprising forming a thermocycling chamber; and arranging an electrode assembly so as to be operable to generate an electric field within the thermocycling chamber.

In some examples, the method may comprise providing a substrate, and arranging the electrode assembly on the substrate. In some examples, the electrode assembly may be in the form of two concentric electrodes as described previously. In some examples, the electrode assembly has a maximum dimension (length or width) of from 5 to 200 μm, for example from 5 to 100 μm, for example from 10 to 100 μm. In some examples, the substrate may be etched to provide one or more recesses or traces into which the electrode assembly can be inserted or printed.

In some examples, forming a thermocycling chamber may comprise forming or providing a well plate or cover layer having one or more wells or thermocycling chambers, and arranging the well plate or cover layer on the substrate so as to align a well or thermocycling chamber with at least one electrode of an electrode assembly on the substrate. In some examples, the well of a well plate comprises an open channel, with the well plate being bonded to the substrate (for example comprising one or more functional layers as described herein), so that the upper surface of the substrate forms the bottom of the well or thermocycling chamber. In some examples, the thermocycling chamber has an internal diameter of from 5 to 200 μm, for example from 5 to 100 μm, for example from 10 to 100 μm.

In some examples, forming a thermocycling chamber comprises arranging at least one electrode of an electrode assembly on a substrate, and spinning or sputtering at least one of a dielectric layer and a hydrophobic layer on the at least one electrode and substrate, and bonding a well plate to the at least one of a dielectric layer and a hydrophobic layer. It will be understood that where both the dielectric layer and the hydrophobic layer are present, it is preferred for the hydrophobic layer to be uppermost, that is in contact with a reaction liquid being amplified in the thermocycling chamber.

For example, the substrate may be provided with at least one electrode of the electrode assembly, with a hydrophobic coating overlying the at least one electrode and substrate, with the well plate or cover layer bonded to the hydrophobic coating and the hydrophobic coating and the walls of the well forming the thermocycling chamber.

In some examples, at least one electrode of an electrode assembly is provided on a substrate of the PCR system as described, and a second electrode of the electrode assembly is provided into the thermocycling chamber through an opening, such as a needle electrode extending into the thermocycling chamber.

In some examples, the thermocycling chamber may be provided with one or a plurality of capillary pressure barriers, for the purpose of controlling infilling of a liquid volume into the thermocycling chamber, as described above. In some examples, a second electrode assembly, for example a plurality of printed electrical traces may be provided on an internal surface of the thermocycling chamber, for example a substrate of the PCR system as described, for the purpose of controlling infilling of a liquid volume and/or providing heat to the liquid volume during thermocycling, and/or for measuring the temperature of the thermocycling chamber, transduced by changes in resistance. The second electrode assembly may be independently operable from the electrowetting electrode assembly.

In some examples, the method comprises providing an optical sensor configured to obtain optical signals from the thermocycling chamber. In some examples, the optical sensor is a fluorescent sensor. In some examples, the optical sensor is directly integrated into the thermocycling chamber, for example into a wall or cover of the thermocycling chamber or is located elsewhere in the system but configured to receive signals from the thermocycling chamber.

### Method of Performing PCR

In some examples, there is provided a method of performing PCR, comprising:
introducing a liquid volume into a thermocycling chamber of a PCR system, wherein the liquid volume comprises a nucleic acid sample suspected of containing a nucleic acid of interest and the thermocycling chamber comprises an electrode assembly configured to generate an electric field within the thermocycling chamber;
applying a first voltage across the electrode assembly to cause the liquid volume to electrowet and form a thin film within the thermocycling chamber;
subjecting the liquid volume to amplification by polymerase chain reaction;
applying a second voltage across the electrode assembly to reverse the electrowetting and cause the liquid volume to form a droplet; and
detecting an optical signal from the droplet.

In some examples, the PCR system is as described herein. In some examples, the liquid volume introduced into the thermocycling chamber comprises an aqueous solution of a nucleic acid sample along with PCR reactants and reagents. In some examples, the liquid volume may be referred to as a test solution or a PCR mixture. In some examples, the liquid volume comprises one or more pairs of PCR primers complementary to a nucleic acid sample of interest, a polymerase, dNTPs and salts such as MgCl₂. Suitable polymerases include the thermostable polymerases Taq, Bst and Pfu. In some examples, the test solution comprises the four standard dNTPs, i.e. dGTP, dCTP, dATP and TTP. In some examples, the liquid volume also contains one or more reporter molecules that permit monitoring of the amplification by optical means. In some examples, the one or more reporter molecules comprise non-specific fluorescent dyes, such as SYBR Green, which intercalates into any double-stranded DNA, leading to an increase in fluorescence as more double-stranded DNA is produced. Other reporter molecules include target-specific fluorescent reporter molecules, such as the TaqMan hydrolysis probes of target-specific nucleic acids labelled with fluorescent reporter and quencher, with the probe being hydrolyzed by the exonuclease activity of the Taq polymerase, releasing the reporter from the quencher and again leading to an increase in fluorescence. The fluorescent reporter may also be linked to a primer to be used in the PCR amplification, such as in the Scorpion® system, in which a single-stranded bi-labeled fluorescent probe sequence forming a hairpin-loop conformation with a 5′ end reporter and an internal quencher Is directly linked to the 5′ end of a primer via a blocker (which prevents the polymerase from extending the primer). In the beginning, the polymerase extends the primer and synthesizes the complementary strand of the specific target sequence. During the next cycle, the hairpin-loop unfolds and the loop-region of the probe hybridizes intramolecularly to the newly synthesized target sequence. Now that the reporter is no longer in close proximity to the quencher, fluorescence emission may take place. The fluorescent signal is detected and is directly proportional to the amount of amplified nucleic acid.

In some examples, the test solution may be prepared by combining the nucleic acid sample, first and second oligonucleotides forming a primer pair complementary to the nucleic acid of interest, the dNTPs, polymerase and buffer/salts. A PCR “Master Mix” may be added to the test solution before or after the nucleic acid sample has been dissolved or dispersed. A PCR Master Mix is a mixture of PCR reagents, already at optimized concentrations, which can be readily aliquoted and added to the test solution. The Master Mix usually comprises the DNA elongation enzyme (e.g. a polymerase), the dNTPs, MgCl₂ as an enzyme co-factor (although other co-factors, such as MgSO₄ may be used with certain enzymes), all dissolved in an aqueous buffer. The Master Mix may also include a reporter molecule, such as a fluorescent dye as described herein. The LightCycler® 480 SYBR Green I Master Mix includes a polymerase, co-factor, dNTPs and SYBR Green I in a buffered solution, meaning that only the nucleic acid sample and, if appropriate, a primer or primer pair need to be added. However, the reporter molecule may also be added separately.

In some examples, the test solution comprises a plurality of oligonucleotide or primer pairs, each complementary to a different nucleic acid of interest. In these examples, a multiplexed PCR analysis is enabled.

In some examples, the liquid volume has a volume of less than 100 UL, for example less than 50 μL, for example less than 25 μL, for example less than 10 μL, for example about 5 μL. In some examples, the liquid volume has a volume of greater than 5 μL, for example greater than 10 μL, for example greater than 25 μL, for example greater than 50 μL, for example about 100 μL.

In some examples, the test solution comprises a nucleic acid sample obtained from a subject. In some examples, the nucleic acid sample may comprise a nucleic acid for analysis and is to be amplified in a method as described herein. In some examples, the nucleic acid sample may comprise a plurality of nucleic acids for analysis which are to be amplified in a method as described herein. In some examples, the test solution is suspected of comprising one or a plurality of nucleic acid sequences of interest. In some examples, the nucleic acid sample is obtained from one or more of a blood sample, a tissue sample, a saliva sample or mucosal sample. In some examples, the nucleic acid sample is obtained using a swab. In some examples, the nucleic acid sample is isolated from the bodily fluid or tissue via which it was obtained. In some examples, the nucleic acid sample is not isolated from the bodily fluid or tissue via which it was obtained. In some examples, the nucleic acid sample obtained from a subject is incorporated into a test solution with or without any isolation or preparation. In some examples, the nucleic acid sample obtained from a subject is dissolved or dispersed in an aqueous solution, thus forming a test solution. In some examples, a primer pair complementary to the nucleic acid sequence of interest, a polymerase, and mix of dNTPs may also be added to the test solution before or after the nucleic acid sample has been dissolved or dispersed.

In some examples, the liquid volume is introduced into the thermocycling chamber using one or more capillary pressure barriers oriented perpendicular to the direction of flow as described herein. In some examples, the liquid volume is caused to flow until it reaches a first capillary pressure barrier, at which point a pulse of heat is provided to the first capillary pressure barrier, thereby raising the temperature of the liquid volume at the capillary pressure barrier enough to breach the barrier and continue progress. In some examples, the thermocycling chamber is provided with a plurality of capillary pressure barriers and a pre-programmed series of thermal pulses are provided to the plurality of capillary pressure barriers. In some examples, the series of thermal pulses are timed so as allow liquid to propagate from one capillary pressure barrier to the next capillary pressure barrier, but not earlier.

Once the liquid volume has been introduced into the thermocycling chamber, a first voltage may be applied to cause the liquid volume to electrowet and form a thin, substantially wetted, film within the thermocycling chamber.

In some examples, the method relies on an electrowetting on dielectric phenomenon (“EWOD”) in order to transition the reaction liquid volume between a thin film state and a droplet state. In some examples, a first electrode of the electrode assembly is grounded and a first voltage (e.g., Vbias) is applied across the electrode assembly/to a second electrode of the electrode assembly. The electric field generated spans the electrodes of the electrode assembly, and the force of the electric field causes the liquid volume to spread, or be pulled, into a thin film state, confined by the electrode surface and the walls of the well. In some examples, the first voltage applied comprises a voltage in the range of from 1 V to 1000 V, depending on usage. In some examples, the first voltage may be in the range of from 1-2 V, such as when thin dielectric and/or hydrophobic coatings are used, or electrowetting-on-conductor applications. In some examples, the first voltage may be in the range of from 200 V to 1000 V, for example 250 V to 300 V for electrowetting on thicker dielectric coatings. The applied voltage may be AC or DC. In some examples, the applied voltage may be an AC voltage of 250-300 V with a frequency of 1000 Hz. It will be understood that the voltage and frequency required to electrowet a liquid for any given system can be readily determined.

In some examples, subjecting the liquid volume to amplification by polymerase chain reaction comprises thermocycling the electrowetted liquid volume, i.e. when the liquid volume is in a substantially wetted or thin film form caused by the first voltage applied across the electrodes. In some examples, the method comprises subjecting the liquid volume to amplification by polymerase chain reaction by performing one or more cycles of PCR amplification with the liquid volume in the thin film state prior to any optical detection such as fluorescence detection. In some examples, the one or more cycles of PCR amplification are performed by heating the liquid volume using an electrode assembly as described herein as the second electrode assembly. In these examples, the electrode assembly may comprise one or more electrical traces printed on the substrate on which the thermocycling chamber is provided. It will be appreciated that other heating forms are possible, and that this is provided only by way of example. For example, in some examples, subjecting the liquid volume to amplification by polymerase chain reaction may comprise performing isothermal PCR amplification with the liquid volume in the thin film state prior to any optical detection such as fluorescence detection.

Due to the liquid volume being in the thin film state, heating and cooling rates of the liquid volume are maximised, leading to rapid thermocycling of the liquid volume and rapid amplification of product. Thus, when the liquid volume is in the thin film state, one or more cycles of PCR amplification can be performed much quicker than when the liquid volume forms a bead or droplet.

In some examples, the liquid volume in the thin film state has a thickness or depth of no more than about 400 μm, for example no more than about 300 μm, for example no more than about 250 μm, for example no more than about 200 μm, for example no more than about 150 μm, for example no more than about 100 μm, for example no more than about 50 μm, for example no more than about 25 μm, for example no more than about 20 μm, for example no more than about 10 μm, When a film of such dimensions is thermocycled, heating and cooling rates of 1000° C./second can be achieved. Thus, a single thermal cycle of PCR amplification comprising denaturation, annealing and chain extension is limited by enzyme activity, not thermocycling rates, and thus be as quick as about 0.05 seconds.

In some examples, at an appropriate or desired time, for example after or during one cycle of PCR amplification/thermocycling, or after more than one cycle of PCR amplification, for example after a predetermined number of cycles, the electrodes of the electrode assembly are all grounded, resulting in the turning off of the electric field. In other words, at an appropriate or desired time during the cycles of amplification, a second voltage, for example an almost zero voltage is applied, resulting in the substantial turning off of the electric field. It will be appreciated that it is not necessary to completely switch off or reduce the electric field to zero in order to change the shape of the liquid volume from wetted film to a form resembling a droplet. Thus, references herein to a “second voltage” are to be construed as encompassing a zero voltage as well as any voltage that causes the transition of a reaction volume of liquid from a substantially wetted film to any shape between a wetted film and a sphere or spherical-like droplet. Once the electric field is turned off, or sufficiently reduced, the surface tension of the liquid volume on the electrode surface (or dielectric/hydrophobic coating as the case may be) causes the liquid volume to retract on itself and form a bead, or droplet type shape. The shape and topography of the electrodes of the electrode assembly may determine a shape of the liquid volume in either thin film or droplet state.

In some examples, the voltage is oscillated between the first voltage and the second voltage at a frequency of up to 1000 Hz, for example up to 500 Hz, for example up to 250 Hz, for example up to 200 Hz, for example up to 150 Hz, for example about 100 Hz. That is, the first voltage and the second voltage may be oscillated at a frequency of 100 Hz. It will be appreciated that the frequency of oscillation between the first voltage and the second voltage can be controlled to coincide with or be compatible with heating and cooling rates in the thermocycling process. In some examples, the second voltage is applied during at least one amplification cycle to detect an optical signal from the droplet during the amplification cycle. In some examples, the second voltage comprises a zero or near zero voltage.

FIGS. 1C and 1D show a thermocycling chamber 102 of a PCR system 100 including a liquid volume 112. In these Figures, liquid volume 112 is shown as a thin film, spread out on the lower surface of thermocycling chamber 102. FIG. 1C shows electrodes 104 and 106, while FIG. 1D simply shows substrate 108 for convenience. In these Figures, a first voltage has been applied across the electrode assembly comprising concentric electrodes 104 and 106, causing electrowetting of liquid volume 112 resulting in the thin film seen in FIG. 1D. In this wetted state, the surface area to volume of the liquid droplet is optimised for rapid thermocycling.

FIGS. 1E and 1F show the same system as FIGS. 1C and 1D, but in the state in which the electric field has been substantially switched off by applying a second voltage which may be near zero or zero. As can be seen, in the absence or near absence of an electric field liquid volume 112 has retracted, or beaded up, to form an almost hemispherical droplet. In the example shown in FIG. 1E, liquid volume 112 is wholly within the boundary formed by electrode 104. In this droplet state, enhanced fluorescence detection is possible due to the etendue of the fluorescent reporter molecule.

In some examples, a magnitude of the electric field may be based on a cleanliness level of a hydrophobic coating and the voltage applied to or across the electrode assembly. The cleanliness level is based on the amount of contamination in a surface of the hydrophobic coating. Contamination may be in the form of micro-sized (and smaller) particles, nonvolatile residue, metals, toxic chemicals, and other impurities deposited on the surface of the hydrophobic coating. The cleanliness level of the hydrophobic coating is set to a level sufficient to enable the liquid volume to transition between thin film and droplet states such as in response to the electric field applied.

Monitoring the shape or form of the liquid volume to determine if it is in a thin film or droplet-like state can be performed optically, or by measuring impedance between an electrically ground conducting top layer of the thermocycling chamber and the electrodes of the electrode assembly.

In some examples, a portion of oil is provided into the thermocycling chamber. The function of the oil is to prevent evaporation of any liquid from the liquid volume during amplification if the thermocycling chamber is an open well as described previously. In some examples, light mineral oils such as a white oil are provided. In some examples, the volume of oil provided is in excess of the volume of the liquid volume containing the PCR mixture. In some examples, sufficient oil is provided to completely envelop the liquid volume.

While the main function of the electrowetting is to oscillate the liquid volume between a wetted thin film state for rapid thermocycling and a droplet state for sensitive fluorescence-based amplicon detection by modulating the ratio of surface area to volume, the oscillation also facilitates mixing of reagents within the liquid volume for the thermocycling, as well as promoting thermal uniformity within the liquid volume.

An optical sensor is configured to obtain optical signals from the thermocycling chamber. In some examples, either during or at the end of each cycle of thermocycling, the optical sensor, for example a fluorescence sensor, is used to detect and measure the fluorescence level. In some examples, the fluorescence sensor detects and measures the fluorescence level after each thermocycle, or after 5 thermocycles, or after 10 thermocycles, or any number of cycles as required. If a nucleic acid of interest is present in the sample, it will be amplified through the thermocycling, using the complementary oligonucleotide primer pair. Since the amplification of that particular nucleic acid of interest takes place in the thermocycling chamber, measurement of any presence or increase in fluorescence is an indication that the nucleic acid of interest was present in the sample or test solution. The sooner that a positive result (via fluorescence detection) confirms that a nucleic acid of interest is present in a test solution, the quicker the overall test time.

The present invention enables a simple, rapid point-of-care diagnostics system that can accurately and simultaneously screen for nucleic acid sequences of interest.

While the apparatus, methods and related aspects have been described with reference to certain examples, it will be appreciated that various modifications, changes, omissions, and substitutions can be made without departing from the spirit of the disclosure. It is intended, therefore, that compositions, methods and related aspects be limited only by the scope of the following claims. Unless otherwise stated, the features of any dependent claim can be combined with the features of any of the other dependent claims, and any other independent claim.

1. A PCR system, comprising:
a thermocycling chamber comprising an electrode assembly configured to generate an electric field and cause reversible electrowetting of a PCR mixture within the thermocycling chamber; and
an optical sensor configured to obtain optical signals from the thermocycling chamber.

2. The PCR system according to claim 1, wherein the electrode assembly comprises two concentric electrodes provided on a substrate on which the thermocycling chamber is disposed.

3. The PCR system according to claim 1, wherein the optical sensor is a fluorescence sensor.

4. The PCR system according to claim 1, wherein the electrode assembly comprises a hydrophobic coating of PTFE, cyanoethyl pullulan (CEP), or octadecyltrichlorosilane.

5. The PCR system according to claim 1, wherein the electrode assembly comprises a dielectric coating of polyimide, SU-8, silicon oxide, silicon nitride, aluminium oxide, aluminium nitride or any combination/stack thereof.

6. The PCR system according to claim 1, wherein the PCR system comprises a plurality of thermocycling chambers and the plurality of thermocycling chambers are independently operable.

7. The PCR system according to claim 1, wherein the thermocycling chamber fluidically isolated from any other thermocycling chamber that may be present on the substrate.

8. The PCR system according to claim 1, wherein the thermocycling chamber is provided with one or more capillary pressure barriers on an internal surface thereof.

9. A method of manufacturing a PCR system, comprising:
forming a thermocycling chamber; and
arranging an electrode assembly so as to be operable to generate an electric field within the thermocycling chamber.

10. A method of performing PCR, comprising:
introducing a liquid volume into a thermocycling chamber of a PCR system, wherein the liquid volume comprises a nucleic acid sample suspected of containing a nucleic acid of interest and the thermocycling chamber comprises an electrode assembly configured to generate an electric field within the thermocycling chamber;
applying a first voltage across the electrode assembly to cause the liquid volume to electrowet within the thermocycling chamber;
subjecting the liquid volume to amplification by polymerase chain reaction;
applying a second voltage across the electrode assembly to reverse the electrowetting and cause the liquid volume to form a droplet; and
detecting an optical signal from the droplet.

11. The method according to claim 10, wherein the second voltage is applied during at least one amplification cycle to detect an optical signal from the droplet during amplification cycle.

12. The method according to claim 10, wherein the second voltage comprises a zero or near zero voltage.

13. The method according to claim 10, wherein subjecting the liquid volume to amplification by polymerase chain reaction comprises thermocycling the electrowetted liquid volume.

14. The method according to claim 10, wherein an oil is provided into the thermocycling chamber.

15. The method according to claim 10, wherein the first voltage and the second voltage are oscillated at a frequency of 100 Hz.
